(19) United States
(12) Patent Application Publication
(10) Pub. No.: US 2009/0136919 A1
(43) Pub. Date: May 28, 2009

(54) METHOD TO RAPIDLY QUANTIFY MYCOBACTERIA IN INDUSTRIAL FLUIDS

(76) Inventors: Michael D. Gernon, Phoenixville, PA (US); Conor M. Dowling, Ambler, PA (US); Burce C. Hemming, Manchester, MO (US)

Correspondence Address: ARKEMA INC.;PATENT DEPARTMENT - 26TH FLOOR, 2000 MARKET STREET, PHILADELPHIA, PA, 19103-3222, US

(21) Appl. No.: 11/577,998

(22) PCT Filed: Oct. 5, 2005
(86) PCT No.: PCT/US05/35881

§ 371 Date: Apr. 26, 2007

Related U.S. Application Data
(60) Provisional application No. 60/623,675, filed on Oct. 29, 2004.

(51) Int. Cl. C12Q 1/68

(52) U.S. Cl. 435/6

(57) ABSTRACT

The invention is a method for detecting and quantifying environmental mycobacteria in industrial fluids comprising extracting bacterial DNA from a sample of an industrial fluid; amplifying the extracted DNA; hybridizing an aliquot of the DNA to two or more amplification primers; and detecting the amplification product.

### FIELD OF THE INVENTION

The invention relates to methods for detecting and quantifying bacteria of the genus Mycobacterium in industrial fluids.

### BACKGROUND OF THE INVENTION

Mycobacteria are aerobic, gram positive, acid-fast bacteria. The cell walls of mycobacteria are rich in mycolic acids, and mycobacteria express a number of genus-specific enzymes that regulate the synthesis and breakdown of mycolic acids (see Y. Yuan, R. E. Lee, G. S. Besra, J. T. Belisle, C. E. Barry III, Proc. Natl. Acad. Sci. USA 92: 6630-6634, 1995; R. Gande, KJ Gibson, A K Brown, K Krumbach, LG Dover, H. Sahm, S. Shioyama, T Oikawa, G. S. Besra, L. Eggeling, J. Biol. Chem. Aug. 11, 2004 Epub). The genus Mycobacterium includes both pathogenic and environmental species. Pathogenic mycobacteria include the causative agents for infectious diseases such as tuberculosis (M. tuberculosis) and leprosy (M. leprae). Other species, which are not considered pathogenic, may become opportunistic pathogens in immunocompromised individuals. These pathogens continue to be important subjects for clinical research with the goal of improving methods of diagnosis and treatment of the diseases caused by these agents. For these clinical and diagnostic studies, pathogenic mycobacteria are generally isolated from clinical specimens such as blood, urine, saliva, feces, skin and other body tissues.

Environmental mycobacteria can be found in natural and “man-made” aquatic environments. Environmental mycobacteria are not agents of infectious diseases or opportunistic pathogens, but have been associated with allergenic and respiratory disorders such as hypersensitivity pneumonitis (H. Rossmore, L. Rossmore, D. Bassett, Lubes ‘N’ Greases 20-27, April 2004; J. O. Falkinham, III, Emerging Infectious Diseases 9: 763-767, 2003). Hypersensitivity pneumonitis has been strongly correlated with chronic exposure to metal working fluids, and environmental mycobacteria such as M. abscessus, M. chelonae, and M. immunogenum have been isolated from metal working fluids in facilities with high incidences of hypersensitivity pneumonitis (R. J. Wallace Jr., Y. Zhang, R. W. Wilson, L. Mann, H. Rossmore, Appl. Environ. Microbiol. 68: 5580-84, 2002; M. D. Gernon and B. C. Hemming, Lubes ‘N’ Greases, December: 14-20, 2003; H. Rossmore, L. Rossmore, D. Bassett, Lubes ‘N’ Greases, April: 20-27, 2004; J. O. Falkinham, III, Emerging Infectious Diseases 9: 763-767, 2003).

As a result of these findings, the metal working industry is actively experimenting with antibacterial treatments to control mycobacteria and protect its employees. In order to determine whether the mycobacterial population is being controlled, however, a method is needed to quantify the number of mycobacteria present in an industrial fluid before and after treatment. Traditionally, mycobacterial infection or infestation has been determined by culturing the bacteria and then staining for gram positive bacteria. However, mycobacteria grow slowly and this method requires 4-10 days to accomplish. In addition, the method is not quantitative and will only detect living bacteria. Since even dead mycobacteria can be allergens, a more efficient method is needed which will quantify the total number of mycobacteria, living and nonliving, in an industrial fluid. The invention described herein is a method to rapidly and accurately quantify the total amount of mycobacteria present in an industrial fluid.

The polymerase chain reaction (PCR) is a widely used method for the rapid analysis and quantification of DNA in clinical and research samples. The method relies on the rapid amplification of specific segments of DNA and means to detect and quantify the amplification products. The PCR technique was first described in 1986 (Mullis K., Faloona F., Scharf S., Saiki R., Horn G., Erlich H., Cold Spring Harb. Symp. Quant. Biol. 51: 263-73, 1986), is continually being improved, and is very well known to those of skill in the art (see U.S. Pat. Nos. 4,683,195; 4,683,202; 4,800,159; 4,965,188). Basic PCR methods have previously been employed to diagnose mycobacterial infections in clinical samples (U.S. Pat. No. 5,494,796 (Spears); and to detect mycobacterial ribosomal RNA in clinical samples (U.S. Pat. No. 5,422,242 (Young)).

Real-time PCR is a modification of the basic PCR methods which allows the amplified product to be detected and quantified while the PCR proceeds. Quantitative, real-time PCR was first used to detect mycobacterium in metal working fluids by M. D. Gernon and B. C. Hemming, Lubes ‘N’ Greases, December: 14-20, 2003. The technique is easy to perform, accurately quantifies the number of mycobacterial cells in a fluid sample, can be applied to any type of industrial fluid sample, and can be performed within 24 hours, allowing population changes in a fluid sample to be tracked over time or in response to antibacterial or decontamination treatments or other changes in the fluid environment.

### SUMMARY OF THE INVENTION

A method for detecting and quantifying environmental mycobacteria in industrial fluids comprising: (a) extracting bacterial DNA from a sample of an industrial fluid; (b) amplifying the extracted DNA, the amplifying step comprising: hybridizing an aliquot of the DNA to two or more amplification primers, wherein the primers are complementary to one or more nucleotide sequences specific to the genus Mycobacterium; amplifying a segment of the mycobacterial DNA defined by the hybridized amplification primers by extending the hybridized amplification primers on the segment of mycobacterial nucleic acid to produce an amplification product; and (c) detecting the amplification product.

### DETAILED DESCRIPTION OF THE INVENTION

Basic PCR methods are well known in the art and have been frequently described in laboratory protocol manuals such as Molecular Cloning: A Laboratory Manual, J. Sambrook, D. W. Russell, Cold Spring Harbor Laboratory, 3d Edition, 2001, 999 pp; and Current Protocols in Molecular Biology, Fred M. Ausubel, Roger Brent, Robert E. Kingston, David D. Moore, J. G. Seidman, John A. Smith, Kevin Struhl, Eds., John Wiley & Sons 1998. One modification to the basic PCR method is the inclusion of fluorescent or otherwise detectable dyes or probes that associate with the DNA product as it is amplified in a manner proportional to the amount of DNA synthesized. By measuring the fluorescence associated with the amplification product, the amount of amplified DNA can be determined as the amplification reaction proceeds. Sample analysis occurs concurrently with amplification in the same tube within the same instrument. This technique is known as “real-time” PCR.

Methods and means for performing real-time PCR are known and readily available in the art. Examples of DNA dyes and probes include ethidium bromide, SYBR® Green (Molecular Probes, Inc., Eugene Oreg.), TaqMan®, (Applied Biosystems, Foster City, Calif.), Molecular Beacons, and Scorpions™. References describing real-time PCR methods include U.S. Pat. No. 6,387,621 (Wittwer); U.S. Pat. No. 6,730,501 (Eyre); Higuchi R, Fockler C, Dollinger G, Watson R, Biotechnology 11: 1026-30, 1993; Wittwer C T, Herrmann M G, Moss A A, Rasmussen R P, Biotechniques 22: 130-31, 1997; Ririe K M, Rasmussen R P, Wittwer C T, Anal. Biochem. 245: 154-60, 1997; Morrison T B, Weis J J, Wittwer C T, Biotechniques 24: 954-58, 1998. Real time PCR requires thermal cycling equipment which is capable of measuring the dye or probe associated with the amplification product as the amplification reaction proceeds. Such equipment is commercially available from several sources, such as Applied Biosystems 7300 and 7500 Real-Time PCR systems, Biorad MyiQ Single-Color Real-Time PCR Detection System, Biorad iCycler, Cepheid SmartCycler Systems, and Stratagene MX3000P™ Real-Time PCR System. Kits containing all the reagents necessary for extracting and/or amplifying DNA in basic and real-time PCR are commercially available, for example, Clontech Advantage™ PCR Kits; Qiagen QuantiTect SYBR Green PCR and RT-PCR kits; Stratagene Brilliant Quantitative PCR and RT-PCR kits. Equipment and kits include instruction manuals which also provide methods for performing real-time PCR.

In PCR the sequence specificity of the amplification product is defined by the oligonucleotide amplification primers selected to initiate the amplification process. When amplification primers are chosen which hybridize only to a unique DNA sequence in the sample DNA, only that unique DNA sequence will be amplified. The DNA sequence selected for amplification may be unique to a single species of organism or may be common to a related group of organisms. By choosing species-specific primers, only DNA found in that species will be amplified. For example, many individual Mycobacterium species can be differentiated based on sequence differences in the 5′ end of the 16s ribosomal RNA gene (Harmsen et al., BMC Infectious Diseases 3: 26, 2003; complete article found at http://www.biomedcentral.com/1471-2334/3/26). If amplification primers are used which hybridize to these specific 16s rRNA gene sequences, the amount of DNA from a particular mycobacterium species in a DNA sample can be calculated. Similarly, when genus-specific primers are selected, the amount of DNA from organisms in that genus in a DNA sample can be determined. To measure the amount of all mycobacteria present in a sample of industrial fluid, mycobacterium-specific sequences of DNA can be selected for amplification. Sequences of the complete genomes for four mycobacterial species and sequences of many mycobacterial genes are filed with GenBank and can be used to identify mycobacterial-specific sequences through comparison with known bacterial DNA sequences.

Alternatively, sequences of genes known to be specific to mycobacterium could be selected. For example, one characteristic of all mycobacteria is the synthesis of mycolic acids. Enzymes involved in the synthesis and breakdown of these acids are unique to mycobacteria, e.g., mycolyltransferase (Gande R, et al., J. Biol. Chem. Aug. 11, 2004, Epub preprint), cyclopropane mycolic acid synthase (Yuan Y, et al., Proc. Natl. Acad. Sci. USA 92: 6630-34, 1995). BLAST analysis of GenBank sequences reveals that the gene sequence for mycolic acid synthase is highly conserved among mycobacterium species. Gene sequences for these and other associated enzymes could provide mycobacterium-specific sequences for primer selection and amplification.

The present invention provides a method to determine the amount of environmental mycobacteria present in a sample of industrial fluid through the use of real-time PCR. Industrial fluids include metal working fluids such as removing fluids (also known as cutting fluids and used in the machining of metals through cutting and grinding operations), forming fluids (used for stamping or drawing of fabricated articles such as cans and wire), protecting fluids (also known as inhibiting solutions and used to coat metal items to prevent corrosion) and treating fluids (all other fluids used in metal working such as fluids used for controlled cooling of cast items during the annealing process); hydraulic fluids such as brake fluids, automatic transmission fluids, and general industrial hydraulic fluids used in heavy machinery; uncured aqueous based liquid coatings such as latex paints, water reducible alkyd paints, aqueous based sealants and aqueous based adhesives; emulsion-type fuels containing water such as low emission emulsion diesel fuels; and hydrocarbon fuels such as gasoline, diesel and kerosene containing micro-droplets of water. Environmental mycobacteria include M. abscessus, M. avium, M. chelonae, M. immunogenum, M. intracellulare, M. kansasii, M. marinum, M. smegmatis, M. ulcerans, and other Mycobacterium species that routinely inhabit aqueous and emulsion-type industrial fluids.

Real-time PCR is a process which concurrently amplifies and detects an amplified target DNA segment. A segment of a mycobacterium-specific DNA sequence, the “target DNA,” is chosen for amplification and oligonucleotide primers that hybridize to each end of this target DNA segment are synthesized. A sample of industrial fluid suspected to contain mycobacteria is subjected to centrifugation or filtration, e.g., microfiltration, nanofiltration, to isolate bacteria from the fluid. However, when the concentration of bacteria in the industrial fluid is high, this isolation step may be omitted. Bacterial DNA is extracted and purified by any acceptable method in the art, such as cell lysis followed by a separation step (e.g., precipitation, filtration, centrifugation, column or microbead purification). The extracted DNA is resuspended and the concentration of DNA in the suspension is measured. An aliquot of the DNA is added to the PCR reagent mixture. The amount of DNA added will vary with experimental parameters, such as volume of the reaction mixture, but is preferably from about 30 ng to about 900 ng. The PCR reagent mixture generally includes the selected amplification primers, a dye or labeled probe which associates proportionately with the amplified DNA product, thermo-stable DNA polymerase, nucleotide triphosphates, and salts.

The reaction is then carried out as an automated process in an apparatus capable of amplification of the selected DNA sequence as well as the detection and quantification of the labeled amplification product as it is synthesized. In this process, the temperature of the reaction mixture is cycled through a denaturing step which converts the sample DNA from double-stranded to single-stranded, a primer annealing step in which the primers hybridize to the target DNA sequence of the sample DNA, and an enzyme reaction step in which the hybridized amplification primers are extended on the target DNA segment to produce an amplification product. With each cycle the number of copies of the target DNA sequence is amplified. As the selected DNA sequence is amplified, the fluorescence of the reaction mixture increases proportionately and is detected and quantified by the apparatus after each cycle. The number of cycles required for the reaction mixture to reach a selected baseline fluorescence value is termed the “threshold cycle count” or “Ct.” The Ct value is inversely proportional to the log of the amount of target DNA initially present. Through the use of standards, a calibration curve can be derived that allows the direct calculation of the starting quantity of target DNA in the DNA sample. The amount of target DNA normally present in a mycobacterium is then used to calculate the number of mycobacteria present in the original sample of industrial fluid. It will be appreciated by those of skill in the art that this calculation closely approximates, but does not provide an exact measure of the number of mycobacteria in the fluid.

Use of the described invention is illustrated by the following examples. Those of skill in the art will appreciate that numerous variations in real-time PCR procedures fall within the scope of the claims that follow the examples. These examples are provided only for illustrative purposes and do not limit the scope of the invention.

### EXAMPLE 1

### Extracting Bacterial DNA from a Sample of Industrial Fluid

To each 10 ml sample of a metal working fluid suspected to contain mycobacteria, 5 ml of water was added and the mixture was centrifuged at 8000 g for 10 minutes at 4° C. to precipitate cells and DNA. All but 2.0 ml of supernatant was decanted and the pellet was then resuspended by vortexing for 1 minute. Subsequently, 1.8 ml of the suspension was transferred to a clean tube and centrifuged at 10,000 g for 30 seconds. Solutions for isolating DNA from the cells were purchased from Mo Bio Laboratories, Inc., Solana Beach, Calif. (Microbial genomic DNA isolation Kit, Cat. # 12224-50), and DNA was isolated following the manufacturer's instructions. The supernatant was decanted, 300 ul of microbead denaturing solution was added, and the pellet was resuspended by vortexing. The suspension was transferred to a microbead tube and 50 ul of Solution M1 (a lysis buffer) was added followed by vortexing to lyse the cells and adsorb solution components other than DNA to the microbeads. Microbead tubes were secured horizontally and subjected to vortexing at maximum speed for 10 minutes. The lysis reaction mixture was centrifuged at 10,000 g for 30 seconds to pellet the microbeads. Supernatant was transferred to a clean tube, microbeads were rinsed once, pelleted and the second supernatant combined with the first. Solution M2 was added and the solution placed in a cold ice block and stored at −20° C. for 5 minutes to precipitate impurities. After centrifugation at 10,000 g for 30 seconds, supernatant was transferred to a clean tube and 900 ul of Solution M3 was added. The mixture was vortexed for 5 seconds then passed through a spin column to collect DNA on the spin filter. The filter was washed with 200 ul of Solution M4 to remove impurities then transferred to a clean tube. The adsorbed DNA was washed off the filter with 50 ul of Solution M5, then centrifuged at 10,000 g for 30 seconds. The filter was then centrifuged again at 10,000 g for 1 minute. A 1 ul aliquot of the final DNA solution was used for nanodrop spectrophotometric analysis to determine the DNA concentration. The DNA sample was stored at −20° C. until PCR was performed.

### EXAMPLE 2

### Amplification of Selected Segment of Target Mycobacterial DNA

PCR was conducted in a total reaction volume of 26 ul in a reaction tube with a total volume of 0.9 ml. The reaction mixture contained 15 μl BioRad IQ SYBR Green Supermix (containing 100 mM KCl; 6 mM MgCl2; 0.4 mM each of dATP, dCTP, dGTP, and dTTP; 40 mM Tris-HCl, pH 8.4; 50 units/ml iTaq DNA polymerse; SYBR green I® (Molecular Probes); and 20 nM fluorescein), 10 ul DNA sample (containing 5 ng to 171 ng DNA), and 1 ul of primer solution (concentration of each primer is 40 pM). Primers were 15 base, single-stranded oligonucleotides obtained from Microbe Inotech Laboratories, Inc., St. Louis, Mo. The purchased primers were described as “genus specific for mycobacterium.” These primers were designed to amplify a segment of the mycobacterial DNA which ranges in size from 80-130 base pairs among different Mycobacterium species.

PCR was carried out in a Biorad iCycler thermal cycler which is equipped for real-time detection of amplification product. The PCR reaction mixture was first heated to 95.0° C. for 3 minutes, then subjected to 40 cycles of 95.0° C. for 10 seconds followed by 53.0° C. for 45 seconds. The PCR apparatus was enabled for real-time detection, quantification, and data collection during these 40 cycles. The PCR reaction mixture was then heated to 95.0° C. for 1 minute, then held at 53.0° C. for 1 minute, followed by 84 ten-second cycles beginning at 53.0° C. and increasing the temperature by 0.5° C. for each successive cycle, reaching a final temperature of 95.0° C. Melt curve (loss of fluorescence as a function of temperature) data collection and analysis were enabled during these 84 cycles. A melt curve is used to determine the homogeneity of the amplification product. For a homogeneous amplification product, loss of fluorescence will occur sharply over a small temperature range. Melt curves for amplification products derived from mycobacterial DNA from industrial fluids were comparable to those obtained with DNA taken from mycobacterial cultures.

### EXAMPLE 3

### Converting Real-Time PCR Data to Number of Mycobacterial Cells in Sample

Known concentrations of Mycobacterium chelonae genomic DNA were amplified concurrently with samples and used as standards to construct a calibration curve that allows direct calculation of the starting quantity (SQ) of target DNA. For this experiment, the calibration equation was Ct=(−6.208)(Log(SQ))+42.3273. SQ=ng mycobacterial target DNA; Ct threshold cycle count, i.e., number of cycles needed to reach selected baseline fluorescence level. The DNA sample was extracted from 10 ml of metal working fluid. After correcting for the loss of 0.2 ml in the first extraction step (pellet from 10 ml resuspended to 2.0 ml and 1.8 ml aliquot taken for further processing), the calculation to determine the amount of target DNA in the metal working fluid sample becomes DNA (ng/ml)=0.555 SQ. An average bacterial cell weighs 2.8×10⁻¹⁴ grams and 3.9% of the weight is DNA. Therefore, the cell count, “CC,” in bacteria/ml will be 5.088×10⁵ (SQ). This formula was used to convert the data from the 40 amplification cycles to the cell counts shown in Table 1. The time required from isolation of DNA to calculation of mycobacteria concentration in the fluid sample is less than 24 hours.

TABLE 1
Number of mycobacterial cells in metal working
fluid based on amplification data.

| | | | |
|---|---|---|---|
| ID | Ct | SQ (ng DNA) | cells per ml fluid |

| | | | | |
|---|---|---|---|---|
| 1 | 36.46 | 9 | (5 ng/ml) | 4.58E+06 |
| 2 | 30.48 | 82 | (45.6 ng/ml) | 4.17E+07 |
| 3 | 31.91 | 49 | (27.2 ng/ml) | 2.49E+07 |
| 4 | 27.95 | 211 | (117.2 ng/ml) | 1.07E+08 |
| 5 | 30.21 | 91 | (50.6 ng/ml) | 4.63E+07 |
| 6 | 26.92 | 308 | (171.1 ng/ml) | 1.57E+08 |

ID = sample identification, Ct = threshold cycle count, SQ = ng mycobacterial target DNA before amplification.

Although the invention is illustrated and described with reference to specific embodiments, the invention is not intended to be limited to the details shown herein. Various modifications may be made in the details within the scope and range of equivalents of the claims without departing from the invention. Having described the invention, we now claim the following and their equivalents.

What is claimed is:

1. A method for detecting and quantifying environmental mycobacteria in industrial fluids comprising:
(a) extracting bacterial DNA from a sample of an industrial fluid;
(b) amplifying the extracted DNA, the amplifying step comprising hybridizing an aliquot of the DNA to two or more amplification primers, wherein the primers are complementary to one or more nucleotide sequences specific to the genus Mycobacterium; amplifying a segment of the mycobacterial DNA defined by the hybridized amplification primers by extending the hybridized amplification primers on the segment of mycobacterial nucleic acid to produce an amplification product; and
(c) detecting the amplification product.

2. The method of claim 1 further comprising quantifying the amount of DNA present in the amplification product.

3. The method of claim 2 further comprising calculating the amount of mycobacterial DNA present in the sample of industrial fluid.

4. The method of claim 1 wherein the industrial fluid is selected from the group consisting of metal working fluids, hydraulic fluids, uncured fluid coatings, uncured fluid sealants, uncured fluid adhesives, stored fluid fuels, fluid lubricants, and paints.

5. The method of claim 1 wherein the industrial fluid is a metal working fluid.

6. The method of claim 1 wherein the industrial fluid is a hydraulic fluid.

7. The method of claim 1 wherein the industrial fluid is a paint.

8. The method of claim 1 wherein the bacteria are isolated from the industrial fluid by a method selected from the group consisting of centrifugation and filtration.

9. The method of claim 1 wherein the amplification product is detected and quantified by real-time PCR.

10. The method of claim 1 wherein the mycobacterial species detected are selected from the group consisting of M. abscessus, M. avium, M. chelonae, M. immunogenum, M. intracellulare, M. kansasii, M. marinum, M. smegmatis, and M. ulcerans.
